(19) United States
(12) Patent Application Publication
(10) Pub. No.: US 2022/0254503 A1
(43) Pub. Date: Aug. 11, 2022

(54) OPHTHALMOSURGICAL CONTROL MODULE DEVICE

(71) Applicant: Carl Zeiss Meditec AG, Jena (DE)

(72) Inventors: Christoph Kuebler, Oberkochen (DE); Susanne Kohlhammer, Blaustein (DE); Markus Neumaier, Aalen (DE)

(21) Appl. No.: 17/732,501

(22) Filed: Apr. 28, 2022

Related U.S. Application Data
(63) Continuation of application No. PCT/EP2020/080249, filed on Oct. 28, 2020.

(30) Foreign Application Priority Data
Oct. 29, 2019 (DE) 10 2019 216 670.9

(51) Int. Cl. G16H 50/30; A61F 9/007; G16H 40/63

(52) U.S. Cl. CPC G16H 50/30; G16H 40/63; A61F 9/007

(57) ABSTRACT

An ophthalmosurgical control module device includes a first convolution function device configured to output a quantity, convolved with a first convolution function, of a time derivative of the first measurement signal, a first comparison device configured to compare the output quantity of the time derivative of the first measurement signal and the first threshold value with each other and output a first comparison result, a second convolution function device, a second threshold value device, a second comparison device configured to output a second comparison result, a first evaluation device configured to receive and evaluate the first comparison result and the second comparison result and output a first evaluation signal, and an activation device configured to receive the first evaluation signal and, in accordance with the first evaluation signal, to make available a control signal for controlling a parameter of an ophthalmosurgical appliance.

### CROSS REFERENCE TO RELATED APPLICATIONS

This application is a continuation application of international patent application PCT/EP2020/080249, filed Oct. 28, 2020, designating the United States and claiming priority to German application DE 10 2019 216 670.9, filed Oct. 29, 2019, and the entire content of both applications is incorporated herein by reference.

### TECHNICAL FIELD

The disclosure relates to an ophthalmosurgical control module device.

### BACKGROUND

There are a number of surgical techniques for treating clouding of the crystalline lens, which is referred to in medicine as a cataract. The most widespread technique is phacoemulsification, in which a thin hollow needle is introduced into the crystalline lens and is induced to make ultrasonic vibrations. In its immediate surroundings, the vibrating hollow needle emulsifies the lens in such a way that the resulting lens particles can be aspirated through a line with a pump. A flushing fluid (irrigation fluid) is delivered during this process, with the aspiration of the lens particles and of the fluid taking place through an aspiration fluid line. When the lens has been completely emulsified and removed, a new artificial lens can be inserted into the empty capsular bag, and so a patient treated in this way can recover good vision.

During the fragmentation of the crystalline lens by a hollow needle vibrating with ultrasound, it is not possible to avoid a relatively large lens particle reaching the tip of the hollow needle during the surgical operation and clogging said needle tip or the aspiration opening of the latter. This state is referred to as occlusion. In such a case, a pump in the aspiration fluid line builds up a suction pressure that is much stronger in comparison with an occlusion-free operation. In addition, a high energy input for movement of the hollow needle can be effected, so that the lens particle clogging the hollow needle is broken to pieces. Alternatively, a reversal of the direction of movement of the pump can also remove the lens particle from the needle tip, and so normal aspiration of the fluid and of the small lens particles can proceed again. At such a moment, an occlusion is therefore broken up, with the previously present high negative pressure decreasing very rapidly. The resulting suction can lead to not only small lens particles and fluid being drawn to the aspiration line, but also part of the capsular bag coming into contact with the hollow needle. If the capsular bag is punctured, this leads to considerable complications for the patient, which complications absolutely have to be avoided.

Moreover, during the suction, a large quantity of fluid can be aspirated from the anterior chamber of the eye, and so there is a risk of the eye collapsing. This too can lead to considerable complications for the patient, which complications absolutely have to be avoided.

The start of an occlusion or the breakup of an occlusion can be identified by detection of measurement signals in an ophthalmosurgical system. For example, a pressure or a volumetric flow in an irrigation fluid line or an aspiration fluid line can be detected. It is particularly expedient if the derivation of a pressure profile or volumetric flow is performed over time, since a change of the signal profile can be rapidly identified in this way. However, a signal profile for pressure and volumetric flow always has a noise, and therefore a derivation of such a signal can lead to high deflections. There is therefore a relatively high probability of a supposedly significant change of pressure or change of volumetric flow being wrongly interpreted. A status description of an ophthalmosurgical system, as determined on this basis, can lead to hectic switching processes and therefore to complications during phacoemulsification.

### SUMMARY

It is an object of the disclosure to provide an ophthalmosurgical control module device with which the probability of a medical complication or risk of injury during phacoemulsification can be reduced. It is a further object of the disclosure to provide an ophthalmosurgical system having such a control module device.

The object is achieved by an ophthalmosurgical control module device and an ophthalmosurgical system as described herein.

According to an aspect of the disclosure, the ophthalmosurgical control module device includes:
a first convolution function device, which is configured to receive, at its input, a first measurement signal of a first measuring device detected on an irrigation fluid line and, at its output, to output a quantity, convolved with a first convolution function, of a time derivative of the first measurement signal, wherein the first convolution function has a first time duration,
a first threshold value device, with which a first threshold value for the time derivative of the first measurement signal can be established,
a first comparison device, which is configured to receive the quantity, output by the first convolution function device, of the time derivative of the first measurement signal and to receive the first threshold value and, after expiration of a predetermined first time point, to compare the output quantity of the time derivative of the first measurement signal and the first threshold value with each other and output a first comparison result,
a second convolution function device, which is configured to receive, at its input, the first measurement signal detected on the irrigation fluid line and, at its output, to output a quantity, convolved with a second convolution function, of a time derivative of the first measurement signal, wherein the second convolution function has a second time duration, which is shorter than the first time duration,
a second threshold value device, with which a second threshold value for the time derivative of the first measurement signal can be established,
a second comparison device, which is configured to receive the quantity, output by the second convolution function device, of the time derivative of the first measurement signal and to receive the second threshold value and, after expiration of a predetermined second time point, to compare the output quantity of the time derivative of the first measurement signal and the second threshold value with each other and output a second comparison result,
a first evaluation device, which is configured to receive and evaluate the first comparison result and the second comparison result and output a first evaluation signal,
an activation device, which is configured to receive the first evaluation signal and, in accordance with the first evaluation signal, to make available a control signal for controlling a parameter of an ophthalmosurgical appliance.

A convolution function device is a device which is configured to effect a convolution of data with a convolution function. A convolution is a mathematical operator which, from two functions, generates a third function (“convolved function”). The control module device is therefore suitable for convolving a measurement signal, which forms a first function, with a convolution function of a convolution function device. The first convolution function has a first time duration. This means that measurement values of the measurement signal, which are available during a first time duration, are convolved with the convolution function. For example, these can be the last 100 measurement values of the measurement signal that were available before the convolution. The second convolution function has a second time duration, which is shorter than the first time duration. This means that measurement values of the measurement signal, which are available during the second time duration, are convolved with the second convolution function. Since the second time duration is shorter than the first time duration, fewer measurement values of the measurement signal are taken into consideration and convolved by the second convolution function. For example, these can be the last 10 measurement values of the measurement signal that were available before the convolution.

According to an aspect of the disclosure, the first convolution function and also the second convolution function have the effect that a quantity of a time derivative of the first measurement signal is output at the output of the first and second convolution function device. If a measurement signal experiences a change in its profile, this can be quickly and clearly identified with a derivation of the signal profile. Since a first convolution function device and a second convolution function device with respective convolution functions of different time duration are used, it is possible, by taking account of a respective threshold value and comparing the convolved signals with the respective threshold values, to identify whether the measurement signal has an interference signal or noise signal and therefore there is also no occlusion or no breakup of an occlusion. It is equally possible to detect the absence of such an interference signal or noise signal, such that a rapid and in particular reliable detection of an occlusion or of the breakup of an occlusion is possible. In accordance with the first evaluation signal, the activation device can then make available a control signal for controlling a parameter of the ophthalmosurgical appliance. Such a parameter can be a pressure generated by a fluid pump in an irrigation fluid line or aspiration fluid line, or a volumetric flow effected by a fluid pump in an irrigation fluid line or aspiration fluid line, or a modified energy supply to piezoceramics of a surgical handpiece. In this way, the probability of a medical complication or a risk of injury during phacoemulsification can be reduced.

According to an exemplary embodiment of the disclosure, the first convolution function and the second convolution function are each point-symmetrical in relation to their respective coordinate origin. This is advantageous since, after the convolution, there is therefore no shift of the absolute quantity of the output value or any preference for individual measurement values of the measurement signal. Moreover, a point symmetry is advantageous for the precision of the determined derivation of the first measurement signal.

The quantity of the second threshold value is typically different than the quantity of the first threshold value. Particularly typically, the quantity of the second threshold value is higher than the quantity of the first threshold value. The second threshold value is processed with a quantity of the second convolution function device, which has a convolution function with a second time duration, the latter being shorter than the first time duration. If the second threshold value is higher than the first threshold value, the quantity output by the second convolution function device must therefore have a higher quantity than the value output by the first convolution function device, in order to reach an intersection point with the second threshold value. Since the second time duration is shorter than the first time duration, fewer measurement values than in the first convolution function are taken into account. The second convolution therefore has to overcome a higher hurdle or a higher threshold value in order to be able to be provided by the comparison device with the assessment “threshold value exceeded”. A very recent interference signal or noise signal therefore has less chance of distorting an evaluation, thereby permitting still more reliable detection of an occlusion or of an occlusion breakthrough.

Typically, the control module device additionally includes:
a third convolution function device, which is configured to receive, at its input, a second measurement signal of a second measuring device detected on an aspiration fluid line and, at its output, to output a quantity, convolved with a third convolution function, of a time derivative of the second measurement signal, wherein the third convolution function has a third time duration,
a third threshold value device, with which a third threshold value for the time derivative of the second measurement signal can be established,
a third comparison device, which is configured to receive the quantity, output by the third convolution function device, of the time derivative of the second measurement signal and to receive the third threshold value and, after expiration of a predetermined third time point, to compare the output quantity of the time derivative of the second measurement signal and the third threshold value with each other and output it as third comparison result,
a fourth convolution function device, which is configured to receive, at its input, the second measurement signal detected on the aspiration fluid line and, at its output, to output a quantity, convolved with a fourth convolution function, of a time derivative of the second measurement signal, wherein the fourth convolution function has a fourth time duration, which is shorter than the third time duration,
a fourth threshold value device, with which a fourth threshold value for the time derivative of the second measurement signal can be established,
a fourth comparison device, which is configured to receive the quantity, output by the fourth convolution function device, of the time derivative of the second measurement signal and to receive the fourth threshold value and, after expiration of a predetermined fourth time point, to compare the output quantity of the time derivative of the second measurement signal and the fourth threshold value with each other and output it as fourth comparison result,
a second evaluation device, which is configured to receive and evaluate the third comparison result and the fourth comparison result and output a second evaluation signal, and
wherein the activation device is configured to receive the second evaluation signal and, in accordance with the first evaluation signal and the second evaluation signal, to make available a control signal for controlling a parameter of an ophthalmosurgical appliance.

It is thus possible to take account also of a second measurement signal, which is detected on an aspiration fluid line. This is advantageous since, with a first evaluation signal and a second evaluation signal, a redundancy is achieved which permits a still more reliable detection of an occlusion or of an occlusion breakthrough.

According to an exemplary embodiment of the disclosure, the first measurement signal or the second measurement signal is a displacement signal of a displacement sensor for determining a fluid level or for determining a membrane position. As an alternative, the first measurement signal or the second measurement signal is a volume signal of a sensor for determining a volume of a fluid chamber. This is advantageous since the control module device can thus be used in a diaphragm pump.

Moreover, the third convolution function and the fourth convolution function can be point-symmetrical in relation to their respective coordinate origin. This is advantageous since, after the convolution, there is therefore no shift of the absolute quantity of the output value or any preference for individual measurement values of the measurement signal. Moreover, a point symmetry is advantageous for the precision of the determined derivation of the first measurement signal.

The quantity of the fourth threshold value is typically different than the quantity of the third threshold value. Particularly typically, the quantity of the fourth threshold value is higher than the quantity of the third threshold value. This avoids a situation where a recent interference signal or noise signal distorts the evaluation.

If the first convolution function is equal to the third convolution function and the second convolution function is equal to the fourth convolution function, the results of the evaluation devices can be very easily compared with one another, thereby allowing an even more reliable conclusion to be drawn regarding the occurrence of an occlusion or of an occlusion breakthrough. This is improved still further if the first threshold value equals the third threshold value, and the second threshold value equals the fourth threshold value.

The ophthalmosurgical system has an ophthalmosurgical control module device as described above. In addition, it also includes an ophthalmosurgical appliance with an irrigation fluid line, a first fluid pump for irrigation, an aspiration fluid line, and a second fluid pump for aspiration.

### BRIEF DESCRIPTION OF THE DRAWINGS

The disclosure will now be described with reference to the drawings wherein:

FIG. 1 shows a schematic representation of an ophthalmosurgical system with an ophthalmosurgical control module device according to an exemplary embodiment of the disclosure;

FIG. 2 shows a schematic representation with diagrams of a respective signal profile of a first measurement device and of a second measurement device, and associated evaluations of the signal profiles;

FIG. 3 shows a schematic representation with diagrams of a respective signal profile of a first measurement device when 15 convolution functions are provided; and

FIG. 4 shows a graph in which the respective time duration of a convolution function is plotted over the number of convolution functions.

### DESCRIPTION OF EXEMPLARY EMBODIMENTS

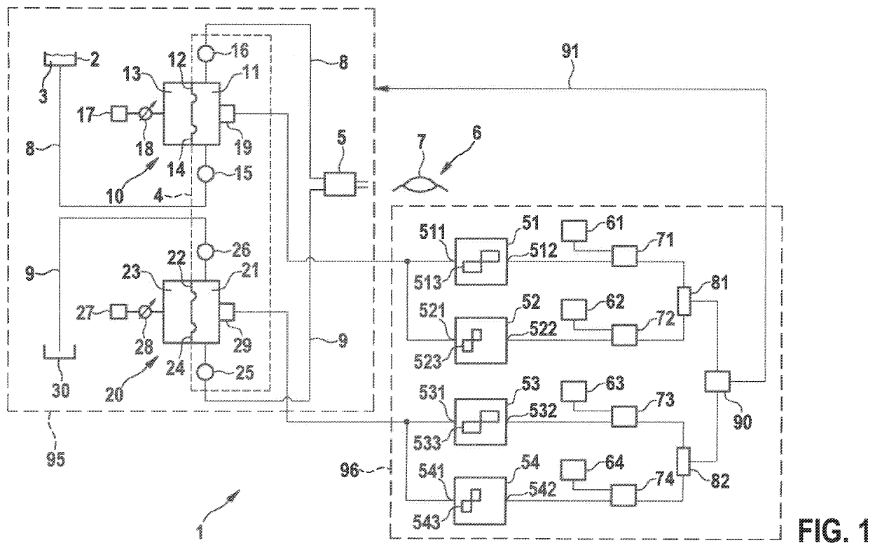

FIG. 1 shows a schematic representation of an ophthalmosurgical system 1 according to an exemplary embodiment of the disclosure. The system 1 includes an ophthalmosurgical control module device 96 and an ophthalmosurgical appliance 95.

The ophthalmosurgical appliance 95 has an irrigation fluid line 8 running from an irrigation fluid container 2, which can be filled with irrigation fluid 3, to a cassette 4. The cassette 4 serves to steer the irrigation fluid 3 to a surgical instrument 5 for an eye 6 that is to be treated. The surgical instrument 5 is suitable for fragmenting a lens 7 of the eye 6, for example by phacoemulsification, it being possible to aspirate the resulting lens particles and the irrigation fluid 3. In addition, the ophthalmosurgical appliance 95 contains a first fluid pump 10 which includes a first pump chamber 11 having a first volume, and a first drive chamber 13 separated therefrom by a first elastic partition element 12 and having a second volume. The first elastic partition element 12 is permanently mounted at its edge 14 in the first fluid pump 10.

The irrigation fluid 3 can be delivered to the first pump chamber 11 by way of the irrigation fluid line 8. The irrigation fluid 3 can flow to a first inlet valve 15, reach the first pump chamber 11 when the inlet valve 15 is in the open position, and leave the first pump chamber 11 again in the event of a first outlet valve 16 being opened. A first drive fluid 17 can, with a proportional valve 18, be fed to the first drive chamber 13 arranged adjacent to the first pump chamber 11. Depending on a differential pressure between the first drive fluid 17 in the first drive chamber 13 and the irrigation fluid 3 in the first pump chamber 11, there is an elastic deformation of the first elastic partition element 12. When the pressure in the first drive chamber 13 is larger than the pressure in the first pump chamber 11, the first volume of the first pump chamber 11 becomes smaller, and at the same time the second volume of the first drive chamber 13 becomes larger. When the inlet valve 15 is closed and the outlet valve 16 opened, irrigation fluid 3 can therefore be conducted out of the first pump chamber 11. The position of the first elastic partition element 12 can be detected with a first measuring device 19, for example a first sensor, which is arranged on the irrigation fluid line 8, for example at the edge of the first pump chamber 11. Here, the irrigation fluid line 8 is understood as a line or a flow path through which irrigation fluid flows. Typically, the first measuring device 19 is an inductive or capacitive displacement sensor.

During the fragmentation of the lens 7 by the surgical instrument 5, the particles are aspirated together with the delivered irrigation fluid. This is effected with a second fluid pump 20. When a second inlet valve 25 is opened, the lens particles and the irrigation fluid contaminated during the fragmentation of the lens 7, which are together then referred to as aspiration fluid, pass along an aspiration fluid line 9 to the second fluid pump 20.

The second fluid pump 20 has a second pump chamber 21 and a second drive chamber 23 arranged adjacent thereto, which chambers are separated from each other with a second elastic partition element 22. The second elastic partition element 22 is permanently joined at its edge 24 to the second fluid pump 20. A second drive fluid 27 can be conveyed to the second drive chamber 23 with a proportional valve 28. When the aspiration fluid flows into the second pump chamber 21 in the event of the inlet valve 25 being opened, said fluid can be conveyed out of the second pump chamber 20 owing to a suitably high pressure in the second drive chamber 23 and thereby elastically deformed second partition element 22 when the second inlet valve 25 is closed and a second outlet valve 26 is opened. The position of the second elastic partition element 22 can be detected with a second measuring device 29, for example a second sensor, which is arranged on the aspiration fluid line, for example at the edge of the second pump chamber 21. The aspiration fluid that is pumped out passes along the aspiration fluid line 9 to an aspiration fluid collection container 30. Here, the aspiration fluid line 9 is understood as a line or a flow path through which aspiration fluid flows. Typically, the second measuring device 29 is an inductive or capacitive displacement sensor.

A first measurement signal can be delivered to the ophthalmosurgical control module device 96 from the first measuring device 19. The first measurement signal reaches an inlet 511 of a first convolution function device 51. The first convolution function device 51 is suitable for generating a convolved signal from measurement values of the first measuring device 19. According to an aspect of the disclosure, the first convolution function device 51 generates a quantity, convolved with a first convolution function 513, of a time derivative of the first measurement signal, which is output at an output 512 of the first convolution function device 51. The first convolution function 513 has a first time duration t1.

The ophthalmosurgical control module device 96 moreover has a first threshold value device 61, with which a first threshold value S1 for the quantity of the time derivative of the first measurement signal can be established. The quantity, output by the first convolution function device 51, of the time derivative of the first measurement signal is then delivered, like the first threshold value S1, to a first comparison device 71 and is compared there after a predetermined first time point. It may then be established that the output quantity of the time derivative of the first measurement signal exceeds or does not exceed the threshold value S1. This result is considered a first comparison result.

The first measurement signal from the first measuring device additionally reaches an input 521 of a second convolution function device 52. Like the first convolution function device 51, the second convolution function device 52 is suitable for generating a convolved signal from measurement values of the first measuring device 19. According to an aspect of the disclosure, the second convolution function device 52 generates a quantity, convolved with a second convolution function 523, of a time derivative of the first measurement signal, which is output at an output 522 of the second convolution function device 52. The second convolution function 523 has a second-time duration t2, which is shorter than the first-time duration t1.

The ophthalmosurgical control module device 96 has a second threshold value device 62, with which a second threshold value S2 for the time derivative of the first measurement signal can be established. The quantity, output by the second convolution function device 52, of the time derivative of the first measurement signal is then delivered to a second comparison device 72. This second comparison device 72 additionally receives the second threshold value S2. In the second comparison device, the quantity of the time derivative of the first measurement signal and the second threshold value S2 are compared with each other after a predetermined second time point. According to an exemplary embodiment, the second time point is identical to the first time point. It may then be established that the output quantity of the time derivative of the first measurement signal exceeds or does not exceed the threshold value S2. This result is considered a second comparison result.

The ophthalmosurgical control module device 96 additionally has a first evaluation device 81, which is configured to receive and evaluate the first comparison result and the second comparison result and output a first evaluation signal. If, for example, both threshold values S1 and S2 are exceeded by the output quantities of the time derivative of the first measurement signal, it can be inferred that the respective measurement value of the displacement signal of the first measuring device 19 has increased at a corresponding speed as the time elapsed increases. The exceeding of the threshold values S1 and S2 signifies that a minimum speed was reached. If there was no occlusion up till then, this signifies that a normal and expected volumetric flow of the irrigation fluid 3 is present in the irrigation fluid line 8.

If, however, after an exceeding of the threshold values S1 and S2, both threshold values S1 and S2 are undershot by the output quantities of the time derivative of the first measurement signal, it can be inferred that the respective measurement value of the displacement signal of the first measuring device 19 has decreased at a corresponding speed as the time elapsed increases. The undershooting of the threshold values S1 and S2 signifies that a minimum speed was no longer maintained. With the evaluation device, it is thus possible to identify that the volumetric flow in the irrigation fluid line has changed, specifically that it has decreased from a higher value to a lower value. The quantities are below the threshold values S1 and S2. This signifies that the flow in the irrigation fluid line is so low that this state can be designated as an occlusion.

The evaluation of whether an occlusion is present or not is then delivered as first evaluation signal to an activation device 90. The activation device 90 can then make available a control signal 91 for controlling a parameter of the ophthalmosurgical appliance 95. For example, the energy supply to the handpiece 5 can be interrupted. Additionally or alternatively, the first fluid pump 10 and/or the second fluid pump 20 can be switched off or their activation can be varied, such that a different volumetric flow and/or pressure can be achieved in the associated irrigation fluid line 8 or aspiration fluid line 9.

If only the second threshold value S2 is undershot, and not the first threshold value S1, it is possible, with the first evaluation device 81, to establish that, although the profile of the time derivative of the first measurement signal is unsteady, this is still not enough to identify a complete occlusion. Such a result could be interpreted to mean that there is a partial occlusion. An associated evaluation signal is then delivered to the activation device 90, which makes available a suitable control signal 91 for controlling a parameter of the ophthalmosurgical appliance 95.

The exemplary embodiment of an ophthalmosurgical control module device 96 shown in FIG. 1 additionally has a third convolution function device 53, which is configured to receive, at its input 531, a second measurement signal of the second measuring device 29 detected on the aspiration fluid line 9 and, at its output 532, to output a quantity, convolved with a third convolution function 533, of a time derivative of the second measurement signal, wherein the third convolution function 533 has a third time duration t3. The third time duration t3 is typically equal to the first-time duration t1.

With a third threshold value device 63, a third threshold device S3 for the time derivative of the second measurement signal can be established. A third comparison device 73 is configured to receive the quantity, output by the third convolution function device 53, of the time derivative of the second measurement signal. The third comparison device 73 is additionally configured to receive the third threshold value S3. If the quantity, output by the third convolution function device 53, of the time derivative of the second measurement signal and the third threshold value S3 are delivered to the third comparison device 73, the latter, after a predetermined third time point, can compare the quantity of the time derivative of the second measurement signal and the third threshold value S3 with each other. It may then be established that the output quantity of the time derivative of the second measurement signal exceeds or does not exceed the threshold value S3. This result is considered a third comparison result. The third time point is typically equal to the first time point.

The second measurement signal from the second measuring device additionally reaches an input 541 of a fourth convolution function device 54. Like the third convolution function device 53, the fourth convolution function device 54 is suitable for generating a convolved signal from measurement values of the second measuring device 29. According to an aspect of the disclosure, the fourth convolution function device 54 generates a quantity, convolved with a fourth convolution function 543, of a time derivative of the second measurement signal, which is output at an output 542 of the fourth convolution function device 54. The fourth convolution function 543 has a fourth time duration t4, which is shorter than the third time duration t3. The fourth time duration t4 is typically equal to the second time duration t2.

A fourth threshold value S4 can be established with a fourth threshold value device 64. This fourth threshold value S4 and the quantity, output by the fourth convolution function device 54, of the time derivative of the second measurement signal can be delivered to a fourth comparison device 74, with which, after a predetermined fourth time point, the output quantity of the time derivative of the second measurement signal and the fourth threshold value S4 are compared with each other. With the fourth comparison device 74, it is possible to establish whether the output quantity of the time derivative of the second measurement signal exceeds or does not exceed the threshold value S4. This result is considered a fourth comparison result. The fourth time point is typically equal to the second time point.

A second evaluation device 82 is configured to receive and evaluate the third comparison result and the fourth comparison result. The following situation may arise: After the ophthalmosurgical appliance 95 was switched on, and therefore after the flow of the irrigation fluid and aspiration fluid, there was still no occlusion. The third comparison result signifies that the threshold value S3 was exceeded, and the fourth comparison result signifies that the threshold value S4 was exceeded. The derivative of the second measurement signal has thus exceeded both threshold values S3 and S4, such that the measurement signal, during the tested time duration after the predetermined third and fourth time points, had a corresponding increase. Therefore, in the aspiration fluid line 9, a volumetric flow was present that can be assessed as being sufficiently high. There is therefore no occlusion in the aspiration fluid line 9.

However, if it is then established that the third comparison result signifies that the third threshold value S3 is undershot, and the fourth comparison result signifies that the fourth threshold value S4 is likewise undershot, the derivative of the second measurement signal has in each case a quantity below the threshold value. This means that the movement of the elastic partition element 22 is still only very slight and there is apparently very little volumetric flow in the aspiration fluid line 9. Such a situation can be evaluated to mean that there is an occlusion. The second evaluation device 82 can then output a corresponding evaluation signal to the activation device 90. With the activation device 90, it is then possible to make available a control signal 91 for controlling a parameter of the ophthalmosurgical appliance 95.

If only the threshold value S4 is undershot, and not the threshold value S3, there appears to be an unsteady signal profile with relatively high fluctuations or with a relatively high noise level. The evaluation device 82 can then conclude that there is still no complete occlusion or that there is a partial occlusion.

It is particularly expedient if the activation device 90 receives the first evaluation signal and the second evaluation signal, since a redundancy can be achieved in this way. The activation device 90 can be configured such that a control signal 91 is supplied to the ophthalmosurgical appliance 95 only when the first evaluation device 81 and the second evaluation device 82 arrive at the same evaluation result.

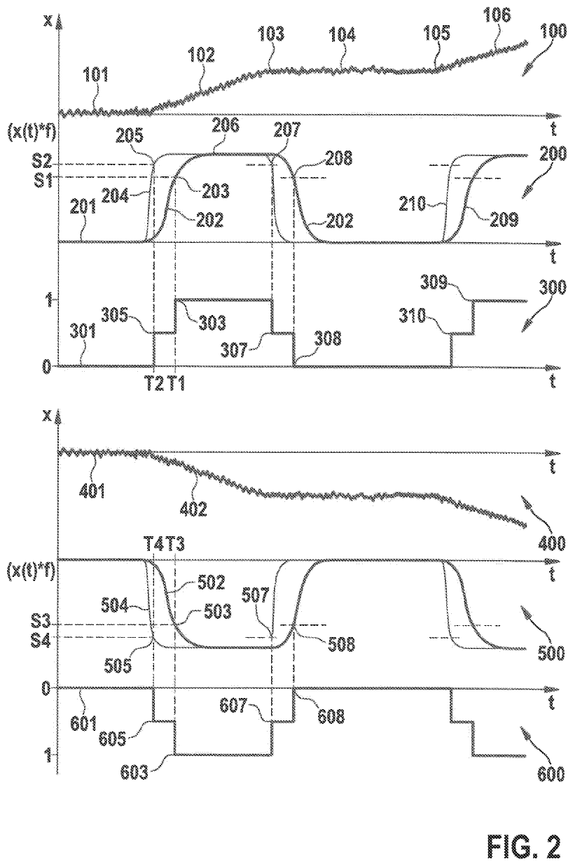

FIG. 2 shows a number of schematic diagrams. The first diagram 100 shows a profile of a displacement x of the elastic partition element 12 of the first fluid pump 10 as a function of the time t. This profile can be determined by the first measuring device 19. It is assumed that the elastic partition element 12 is initially located in a first position (see 101). When the drive fluid 17 is conveyed into the drive chamber 13, the elastic partition element 12 moves in such a way that irrigation fluid 3 is conveyed out of the pump chamber 11. The elastic partition element 12 thus moves closer to the first measuring device 19 (see 102). If an occlusion occurs in the aspiration fluid line 9 (see 103), no further fluid can be fed into the eye, such that the position of the elastic partition element 12 no longer changes (see 104). If the occlusion is broken through (see 105), irrigation fluid can again flow in the irrigation fluid line 8, such that the elastic partition element 12 again moves closer to the first measuring device 19 (see 106). It will be seen in diagram 100 that the signal profile is not completely smooth, and instead it has significant noises.

When the signal of the first measuring device 19 is delivered to the first convolution function device 51 and is there convolved with a first convolution function f1 (see 513) with a first-time duration t1 such that a time derivation of the first measurement signal takes place, a signal (x(t)*f1) can be tapped off at the outlet 512, as is shown in diagram 200. The derivation initially reaches the value zero (see 201) and then rises with a time constant which is assigned to the first convolution function 513 and which, for example, is 0.5 second. This curve profile is designated by 202. The first measurement signal is typically delivered at the same time to the second convolution function device 52, which convolves the signal with a second convolution function f2 (see 523) with a time duration t2, e.g., 50 milliseconds, shorter than the first-time duration t1, such that a signal (x(t)*f2) can be tapped off at the outlet 522. After the pump function of the first fluid pump 10 is started, the time derivative of the first measurement signal achieves a profile (see 204) which has a larger gradient than the time derivative that is achieved after the first convolution function device 51.

The curve 202 intersects a first threshold value S1 at the location 203. The curve 204 intersects a second threshold value S2 at the location 205, the second threshold value S2 being higher than the first threshold value S1. Diagram 300 shows at which time points an intersection point occurs between the curve 202 with the first threshold value S1 and the curve 204 with the second threshold value S2. The curve 202 intersects the first threshold value S1 after expiration of a predetermined first time point. In the situation shown in FIG. 2, the curve 202 intersects the first threshold value S1 at the time point T1. The curve 204 intersects the second threshold value S2 after expiration of a predetermined second time point. In the situation shown in FIG. 2, the curve 204 intersects the second threshold value S2 at the time point T2. Thus, when both threshold values S1 and S2 are exceeded, it is possible to relatively safely conclude that an irrigation fluid is flowing in the irrigation fluid line 8. This conclusion can be drawn on the basis of the curve profile in the third diagram 300. When a threshold value is exceeded, there is an abrupt jump in the curve 301. When the threshold value S2 is exceeded, there is a first jump (see 305), and, when the first threshold value S1 is exceeded, there is a second jump (see 303).

When only the threshold value S2 of the curve 204 is exceeded, and not the threshold value S1 of the curve 202, this is an indication that the rise in the curve 202 has already ended after expiration of the first time point. This shows that the elastic partition element 12 no longer moves in the direction of the first measuring device and the volumetric flow in the irrigation fluid line is interrupted.

However, if a relatively constant volumetric flow is present in the irrigation fluid line 8, the convolved quantity of the derivative of the first measurement signal reaches a constantly high value (see 206). If the first convolution function device 51 is now supplied with a first measurement signal which, after the convolution with the first convolution function device 51, is below the second threshold value S2 (see 207) and also below the first threshold value S1 (see 208), the curve 301 abruptly sinks back again (see 307 and 308). It can be identified from this that a volumetric flow of the irrigation fluid 3 in the irrigation fluid line 8 has decreased.

If the curves 202 and 204 then rise again (see 209 and 210) and exceed the first threshold value S1 and the second threshold value S2, the abrupt elevations (see 309 and 310) in the third diagram 300 show that an occlusion has broken up again.

Still larger certainty is achieved if an analogous evaluation is also made for the second measurement signal of the second measuring device 29. The fourth diagram 400 shows a displacement-time diagram x(t) for the movement of the second elastic partition element 22. From a starting position 401, the second elastic partition element 22 moves ever further away from the second measuring device 29 (see 402). The second measurement signal is delivered to the third convolution function device 53, which convolves the measurement signal with the third convolution function f3 (see 533). The attained quantity (x(t)*f3) for the derivative of the second measurement signal can be tapped off at the output 532 and leads to a curve 502 (see fifth diagram 500). The measurement signal convolved with the fourth convolution function device 54 and the fourth convolution function f4 (see 543) to (x(t)*f4) leads, at the output 542, to a derivation according to curve 504. The curve 502 intersects the third threshold value S3 at 503 after expiration of a predetermined third time point. This intersection point is at the time point T3. The curve 504 intersects the fourth threshold value S4 at 505 after expiration of a fourth time point. This intersection point is at the time point T4. These occurrences can be identified from the sixth diagram 600 (see 603 and 605). If the curves 504 and 503 sink again and drop below a third threshold value S3 and a fourth threshold value S4, the volumetric flow in the aspiration fluid line 9 is apparently so low that an occlusion can be inferred. This can be identified from the profile of the curve 601 in the sixth diagram 600 at the locations 607 and 608.

This information, that an occlusion is present, could already be made possible by evaluation of the first measurement signal. The evaluation also of the second measurement signal permits a redundancy. For example, it can be stipulated that an occlusion is established only when this result is reached by the first measurement signal and by the second measurement signal (AND operation according to Boolean algebra). This increases the reliability of the evaluation of the signal profiles.

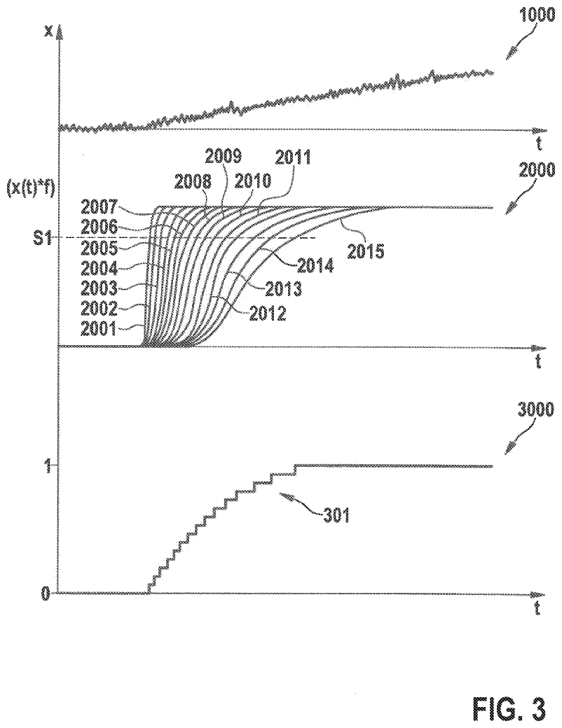

Typically, the control module device has more than two convolution function devices for the evaluation of the first measurement signal. Particularly typically, as the number of the convolution functions applied to the measurement signal increases, the respectively associated time duration of a convolution function decreases. For example, if 15 convolution function devices are provided to which the first measurement signal is delivered, the first convolution function device has a convolution function with a first-time duration, which is the longest time duration compared to the subsequent convolution functions. The second convolution function device has a convolution function with a second-time duration, which is a shorter time duration than the first-time duration, etc. This leads to 15 increments in the profile of the curve 301 (cf. FIG. 3). Diagram 1000 shows a profile of a displacement of the elastic partition element 12 of the first fluid pump 10 as a function of time. Diagram 2000 shows the profile of the quantities, as a function of time, which are output at the outputs of the respective convolution function devices (see curves 2001 to 2015). If in each case a threshold value S1 is exceeded, this is identified by the evaluation device 81, such that the curve 301 is finely stepped (see diagram 3000).

In the exemplary embodiment shown here, each quantity output by the 15 convolution function devices is compared with an identical threshold value S11. According to a further exemplary embodiment, the quantities output by the 15 convolution function devices can be compared with mutually different threshold values S1 to S15.

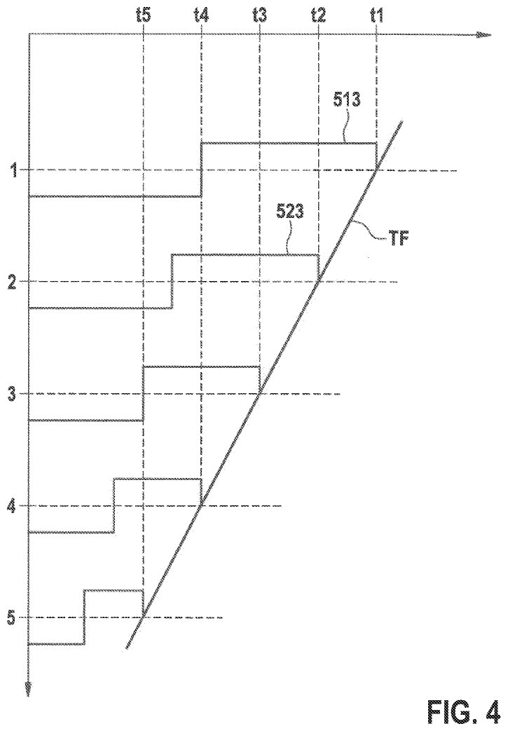

Each of the convolution functions has a time duration in which measurement values of a measurement signal are processed. As the number of convolution functions to which a measurement signal is delivered increases, the time duration associated with these convolution functions decreases. If a respective time duration of a convolution function is plotted over the number of convolution functions, such a time duration curve can adopt a linear, exponential or logarithmic profile. FIG. 4 shows, as an example, a linear profile of such a curve (designated by TF) if 5 convolution functions are provided.

It is understood that the foregoing description is that of the exemplary embodiments of the disclosure and that various changes and modifications may be made thereto without departing from the spirit and scope of the disclosure as defined in the appended claims.

What is claimed is:

1. An ophthalmosurgical control module device, comprising:
a first convolution function device configured to receive, at a first input, a first measurement signal of a first measuring device detected on an irrigation fluid line and, at a first output, to output a first quantity, convolved with a first convolution function, of a time derivative of the first measurement signal, wherein the first convolution function has a first time duration;
a first threshold value device, with which a first threshold value for the time derivative of the first measurement signal can be established;
a first comparison device, configured to receive the quantity, output by the first convolution function device, of the time derivative of the first measurement signal and to receive the first threshold value and, after expiration of a predetermined first time point, to compare the first quantity of the time derivative of the first measurement signal and the first threshold value with each other and output a first comparison result;
a second convolution function device, configured to receive, at a second input, the first measurement signal detected on the irrigation fluid line and, at a second output, to output a second quantity, convolved with a second convolution function, of a time derivative of the first measurement signal, wherein the second convolution function has a second time duration, which is shorter than the first time duration;
a second threshold value device, with which a second threshold value for the time derivative of the first measurement signal can be established;
a second comparison device, configured to receive the second quantity, output by the second convolution function device, of the time derivative of the first measurement signal and to receive the second threshold value and, after expiration of a predetermined second time point, to compare the second quantity of the time derivative of the first measurement signal and the second threshold value with each other and output a second comparison result;
a first evaluation device, configured to receive and evaluate the first comparison result and the second comparison result and output a first evaluation signal; and
an activation device, configured to receive the first evaluation signal and, in accordance with the first evaluation signal, to make available a control signal for controlling a parameter of an ophthalmosurgical appliance.

2. The ophthalmosurgical control module device as claimed in claim 1, wherein the first convolution function and the second convolution function are point-symmetrical in relation to their respective coordinate origin.

3. The ophthalmosurgical control module device as claimed in claim 1, wherein the quantity of the second threshold value is different from the quantity of the first threshold value.

4. The ophthalmosurgical control module device as claimed in claim 1, further comprising:
a third convolution function device, configured to receive, at a third input, a second measurement signal of a second measuring device detected on an aspiration fluid line and, at a third output, to output a third quantity, convolved with a third convolution function, of a time derivative of the second measurement signal, wherein the third convolution function has a third time duration;
a third threshold value device, with which a third threshold value for the time derivative of the second measurement signal can be established;
a third comparison device, configured to receive the third quantity, output by the third convolution function device, of the time derivative of the second measurement signal and to receive the third threshold value and, after expiration of a predetermined third time point, to compare the third quantity of the time derivative of the second measurement signal and the third threshold value with each other and output a third comparison result;
a fourth convolution function device, configured to receive, at a fourth input, the second measurement signal detected on the aspiration fluid line and, at a fourth output, to output a fourth quantity, convolved with a fourth convolution function, of a time derivative of the second measurement signal, wherein the fourth convolution function has a fourth time duration, which is shorter than the third time duration;
a fourth threshold value device, with which a fourth threshold value for the time derivative of the second measurement signal can be established;
a fourth comparison device, configured to receive the fourth quantity, output by the fourth convolution function device, of the time derivative of the second measurement signal and to receive the fourth threshold value and, after expiration of a predetermined fourth time point, to compare the fourth quantity of the time derivative of the second measurement signal and the fourth threshold value with each other and output a fourth comparison result;
a second evaluation device, configured to receive and evaluate the third comparison result and the fourth comparison result and output a second evaluation signal; and
wherein the activation device is configured to receive the second evaluation signal and, in accordance with the first evaluation signal and the second evaluation signal, to make available a control signal for controlling a parameter of an ophthalmosurgical appliance.

5. The ophthalmosurgical control module device as claimed in claim 1, wherein the first measurement signal or the second measurement signal is a displacement signal of a displacement sensor for determining a fluid level or for determining a membrane position, or
wherein the first measurement signal or the second measurement signal is a volume signal of a sensor for determining a volume of a fluid chamber.

6. The ophthalmosurgical control module device as claimed in claim 4, wherein the third convolution function and the fourth convolution function are point-symmetrical in relation to their respective coordinate origin.

7. The ophthalmosurgical control module device as claimed in claim 4, wherein the quantity of the fourth threshold value is different than the quantity of the third threshold value.

8. The ophthalmosurgical control module device as claimed in claim 4, wherein the first convolution function is equal to the third convolution function, and the second convolution function is equal to the fourth convolution function.

9. The ophthalmosurgical control module device as claimed in claim 4, wherein the first threshold value is equal to the third threshold value, and the second threshold value is equal to the fourth threshold value.

10. An ophthalmosurgical system, comprising:
an ophthalmosurgical control module device as claimed in claim 1; and
an ophthalmosurgical appliance comprising:
an irrigation fluid line;
a first fluid pump for irrigation;
an aspiration fluid line; and
a second fluid pump for aspiration.
